(19) United States
(12) Patent Application Publication
(10) Pub. No.: US 2022/0175312 A1
(43) Pub. Date: Jun. 9, 2022

(54) Apparatus and Method For Measurement Of Skin-To-Skin Contact Between Neonate And Parent

(71) Applicant: University of Massachusetts, Boston, MA (US)

(72) Inventors: Apurv Soni, Worcester, MA (US); Nisha Fahey, Worcester, MA (US); Jomol Mathew, Lexington, MA (US)

(21) Appl. No.: 17/593,635

(22) PCT Filed: Mar. 27, 2020
(86) PCT No.: PCT/US2020/025143

§ 371 Date: Sep. 22, 2021

Related U.S. Application Data
(60) Provisional application No. 62/825,545, filed on Mar. 28, 2019.

(51) Int. Cl. A61B 5/00; A61B 5/0205

(52) U.S. Cl. CPC A61B 5/4824; A61B 5/0008; A61B 5/002; A61B 2503/045; A61B 5/02055; A61B 2562/0214; A61B 5/6831

(57) ABSTRACT

An apparatus for measurement of skin-to-skin contact between a neonate and a parent of the neonate may comprise a capacitive touch sensor module configured to receive signals from a first electrode and a second electrode, and produce detected contact information associated with the electrodes. The apparatus may further comprise a temperature module configured to measure a temperature of an object within a field of view of the temperature sensor, and to generate a corresponding temperature code. The apparatus may further comprise a clock module configured to time-stamp each collected data sample with a time-of-day code and store the time-stamped data sample on an associated data storage device. The apparatus may further comprise a processor configured to execute computer code instructions that cause the apparatus to coordinate operation of the capacitive touch sensor module, the temperature module, and the clock module.

### RELATED APPLICATION

This application claims the benefit of U.S. Provisional Application No. 62/825,545, filed on Mar. 28, 2019. The entire teachings of the above application are incorporated herein by reference.

### BACKGROUND

Every year worldwide, nearly 3 million children die during the neonatal period, the first 4 weeks of life. Kangaroo Mother Care (KMC) refers to continuous skin-to-skin contact between the neonate and the parents and positional support for breastfeeding. It is estimated that administering 4 or more hours of KMC per day to more than 90% of premature newborns could halve the global number of neonatal deaths. Recent estimates from India, the epicenter of neonatal mortality, suggest that fewer than 50% of eligible neonates received KMC and only for an average of less than two hours per day. Current guidelines by a nationwide effort in India to promote KMC recommend “initiating KMC as early and for as long as possible.” These nebulous recommendations may cause confusion and cast KMC as an unscientific remedy.

### SUMMARY

The automated measurement of Kangaroo Mother Care (KMC) practices and corresponding changes in newborn physiology can help enhance hospital and home-based KMC practices in India and other countries. The described embodiments are directed to a device for, and method of, measuring skin-to-skin contact between a neonate (also referred to herein as baby or newborn) and a parent of the neonate. The example embodiments described herein may be referred to as the PROMOTE-KMC device. The term “PROMOTE” is derived from “PReventing neOnatal Mortality & mOrbidity using Technology.” The described embodiments are directed to accurately measuring KMC frequency and duration, and continuously recording a range of physiological parameters that are influenced by KMC (e.g., heart rate, respiratory rate, skin surface temperature, sympathetic activity, and newborn positioning). The described embodiments may increase KMC practices by providing real-time feedback on the amount of KMC administered and corresponding beneficial changes in neonate's physiology. Furthermore, data acquired by accurately measuring KMC and associated physiological changes may allow for the future development of evidence-based clinical guidelines.

In one aspect, the invention may be an apparatus for measurement of skin-to-skin contact between a neonate and a parent of the neonate, comprising a capacitive touch sensor module configured to receive signals from a first electrode and a second electrode, and produce detected contact information associated with at least one of the first electrode and the second electrode. The apparatus may further comprise a temperature module having a temperature sensor, the temperature module configured to measure a temperature of an object within a field of view of the temperature sensor, and to generate a corresponding temperature code. The apparatus may further comprise a clock module configured to (i) implement a real-time chronometer, (ii) generate a time-of-day code based on the chronometer, (iii) time-stamp each collected data sample with the time-of-day code, each collected data sample comprising the detected contact information and the temperature code, and (iv) store the time-stamped data sample on an associated data storage device. The apparatus may further comprise a processor and a memory with computer code instructions stored thereon, the memory operatively coupled to the processor such that, when executed by the processor, the computer code instructions cause the apparatus to coordinate operation of the capacitive touch sensor module, the temperature module, and the clock module.

In an embodiment, the touch sensor module, the temperature module, the clock module, the processor and the memory may be disposed within a housing that comprises a device body and device lid, and wherein the device body and the device lid are configured to engage one another to isolate the touch sensor module, the temperature module, the clock module, the processor and the memory from an external environment. The housing may be attached to a flexible belt, the first electrode may be disposed on a first side of the flexible belt, and the second electrode may be disposed on a second side of the belt.

The first electrode may be configured to be in physical contact with skin of the neonate. The second electrode may be configured to be in physical contact with skin of the parent of the neonate. The apparatus may be operative to characterize aspects of skin-to-skin contact between the neonate and the parent of the neonate.

The apparatus may further comprise a wireless transceiver operatively coupled to the processor. The wireless transceiver may be configured to wirelessly communicate information from the apparatus to an external peripheral component. The wireless transceiver may be a Bluetooth Low Energy (BLE) transceiver, a WiFi (e.g., IEEE 802.11 family of protocol standards), or other wireless transceivers based on wireless communication protocols known in the art. The apparatus may further comprise an energy source configured to provide electrical energy to the touch sensor module, the temperature module, the clock module, the processor and the memory. The apparatus may further comprise an inertial measurement unit configured to determine a position of the neonate with respect to one or both of (i) the parent and (ii) a predetermined reference frame.

In another aspect, the invention may be a method of measuring skin-to-skin contact between a neonate and a parent of the neonate, comprising providing a flexible belt for disposing around the neonate, such that a first electrode attached to a first side of the flexible belt is arranged to be in contact with the neonate, and a second electrode attached to a second side of the flexible belt is arranged to be in contact with the parent of the neonate. The method may further comprise (i) receiving, by a capacitive touch sensor module disposed within a housing attached to the flexible belt, information associated with at least one of the first electrode and the second electrode, (ii) measuring, by a temperature module, a temperature of the neonate and generating a corresponding temperature code, (iii) time-stamping, by a clock module, one or both of the information associated with at least one of the first electrode and the second electrode and the temperature code to produce time stamped information, (iv) storing the time stamped information on an associated data storage device.

The method may further comprise (a) measuring physiological parameters, consisting of one or more of (i) heart rate of the neonate, (ii) respiratory rate of the neonate, (iii) sympathetic activity of the neonate, and (iv) positioning of the neonate with respect to the parent of the neonate, (b) time-stamping the physiological parameters; and (c) storing the time stamped physiological parameters on an associated data storage device.

The method may further comprise wirelessly transmitting the time stamped information to a destination that is external to the housing. The method may further comprise displaying, at the destination, a dashboard that presents the time stamped information to a user. The method may further comprise measuring one or both of a temperature and a heart rate of the neonate before an indication of neonate-to-parent skin-to-skin contact, and measuring one or both of the temperature and the heart rate of the neonate after the indication of neonate-to-parent skin-to-skin contact.

In another aspect, the invention may be an apparatus for measurement of skin-to-skin contact between a neonate and a parent of the neonate, comprising a touch sensor that receives signals from a first electrode and a second electrode, and produces detected contact information associated with at least one of the first electrode and the second electrode. The apparatus may further comprise a temperature sensor that measures a temperature of an object within a field of view of the temperature sensor, and generates a corresponding temperature code. The apparatus may further comprise a data accumulator that (i) applies a time-of-day code time stamp to each collected data sample, where each collected data sample comprises the detected contact information and the temperature code, and (ii) stores each time-stamped data sample on an associated data storage device.

The apparatus may further comprise an inertial measurement unit configured to determine a position of the neonate with respect to one or both of (i) the parent and (ii) a predetermined reference frame. The touch sensor, the temperature sensor, and the data accumulator may be disposed within a housing that is configured to isolate the touch sensor module, the temperature module, and the data accumulator from an external environment. The housing may be attached to a flexible belt, the first electrode may be disposed on a first side of the flexible belt, and the second electrode may be disposed on a second side of the belt.

The apparatus may further comprise a wireless transceiver configured to wirelessly communicate information from the apparatus to an external peripheral component. The wireless transceiver may be a Bluetooth Low Energy (BLE) transceiver. The apparatus may further comprise an energy source configured to provide electrical energy to the touch sensor module, the temperature module, and the data accumulator.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1 and 2 show an example of a PROMOTE-KMC device according to the described embodiments.

FIG. 3A shows an example microcontroller module according to the described embodiments.

FIG. 3B shows an example clock module according to the described embodiments.

FIG. 3C shows an example touch sensor module according to the described embodiments.

FIG. 4A shows an example temperature module according to the described embodiments.

FIG. 4B shows an example energy source according to the described embodiments.

FIG. 5 shows a view of an example embodiment of the device body containing several of the components described herein.

FIG. 6A illustrates a smartphone application displaying collected data on a smartphone according to the described embodiments.

FIG. 6B shows an online dashboard displaying collected data according to the described embodiments.

FIG. 7 is a diagram of an example internal structure of a processing system 700 that may be used to implement one or more of the embodiments herein.

FIGS. 8A through 8P depict example instruction code executed to implement the device functions and operations described herein.

FIGS. 9A through 9I depict example instruction code executed to establish a communications link between the PROMOTE-KMC device and a cloud-based reporting application associated with the dashboard described herein.

### DETAILED DESCRIPTION

A description of example embodiments follows.

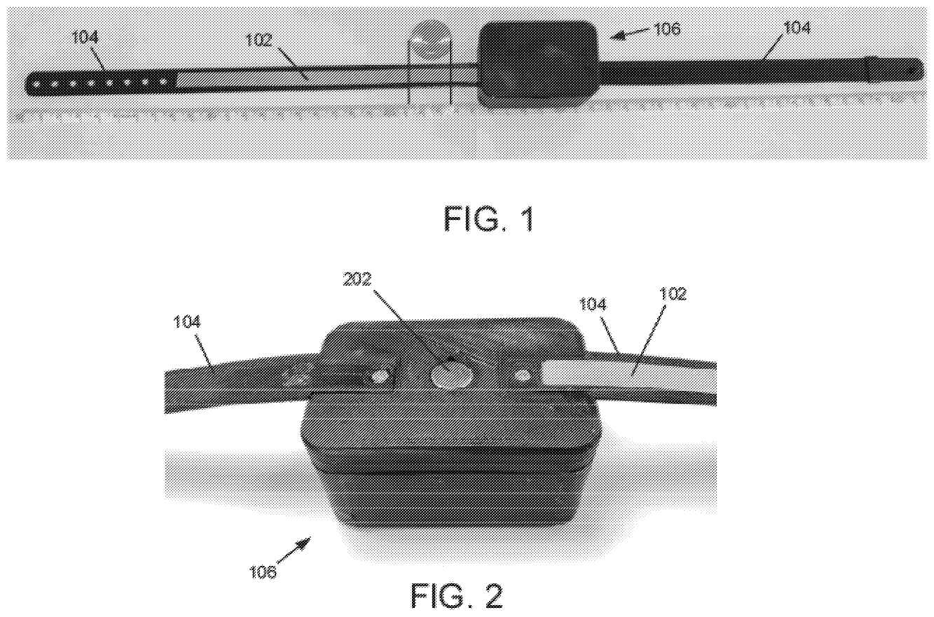

An example embodiment of a PROMOTE- KMC device is shown in FIGS. 1 and 2. FIG. 1 shows the outer (i.e., parent-facing) side of the PROMOTE-KMC device. FIG. 2 shows the inner (i.e., infant-facing) side of the PROMOTE-KMC device. The PROMOTE-KMC device comprises a Capacitive Sensor 102, a Flexible Belt 104, a Device Body 106, and an Infrared Temperature Sensor 202.

The example device body 106 may host various electrical components, for example a Microcontroller with Bluetooth Low Energy (BLE) connectivity, an SD Card +Real Time Circuit Module, a Capacitive Sensor Circuit Breakout module, an Infrared Temperature Sensor module, and a Lithium-Polymer Battery, as described herein. Two capacitive sensors 102, mounted on a flexible belt 104, are connected to the device body 106 with molded copper cables disposed inside the belt.

### PROMOTE-KMC Device Component Specifications

In an example embodiment, the device body was designed using Solidworks CAD, and fabricated with Makerbot Replicator 3D printer. The 3D printer uses polylactic acid (PLA) filament to print the device body. More information on material characteristics and safety document associated with the PLA filament can be found at https://images.makerbot.com/ support/production/SDS-000002ENA.pdf.

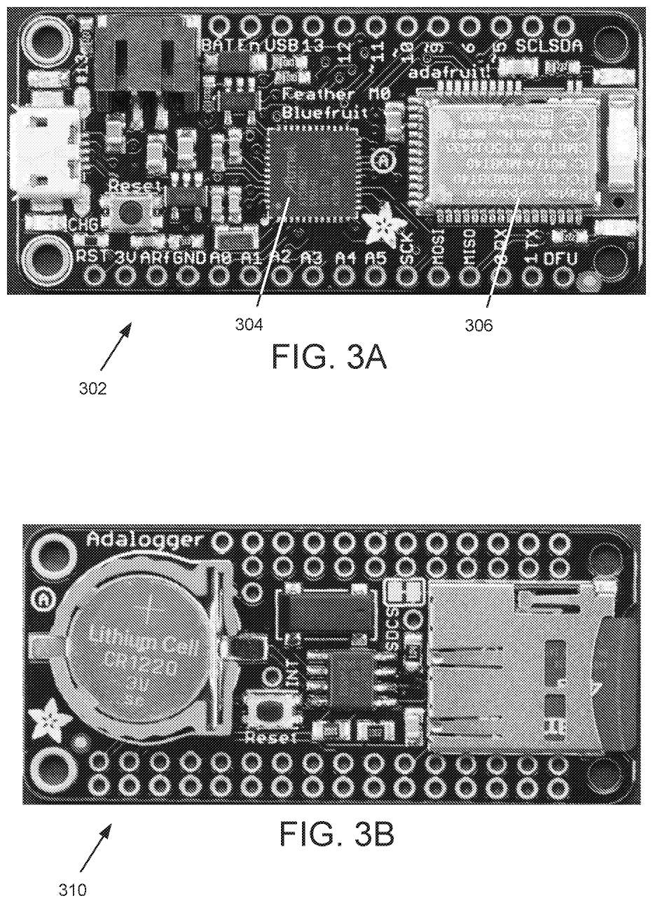

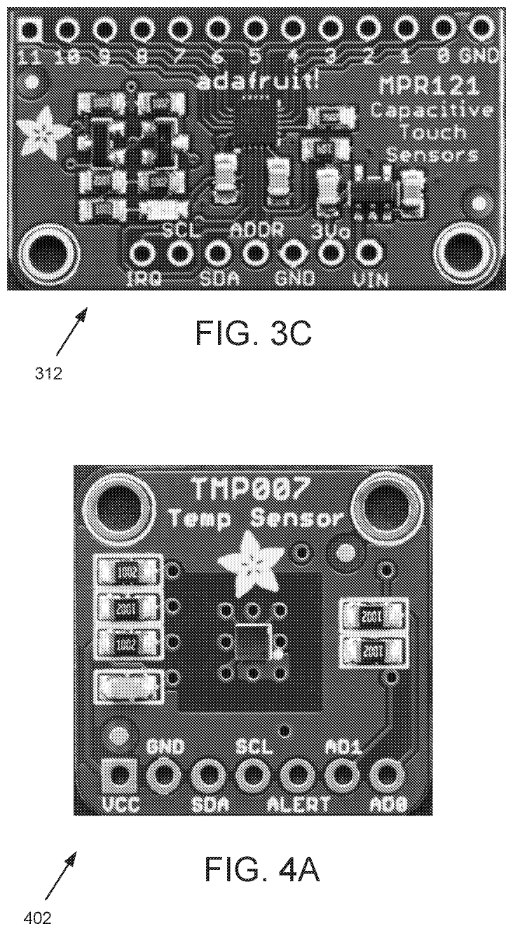

Disposed within the device body 106 of the example embodiment is an Adafruit Feather MO Microcontroller (referred to herein as the “microcontroller module 302”), as shown in FIG. 3A. The microcontroller module 302 is the main central computing unit of the PROMOTE-KMC device. The microcontroller module 302 controls operations performed by the PROMOTE-KMC device, including, for example, data collection from sensors, data transfer, and data storage. The microcontroller module 302 comprises a processor 304, which includes embedded memory configured to store computer code instructions. The memory device is operatively coupled to the processor such that, when executed by the processor, the computer code instructions cause the system to implement the operations described herein.

The microcontroller module 302 may also comprises a Bluetooth Low Energy (BLE) component 306, which facilitates wireless interaction with other peripherals within communication range. Technical specifications of the microcontroller module are set forth below, and additional information regarding the processor 304 may be found at https://cdn-shop.adafruit.com/product-files/2772/atmel-42181-sam-d21 datasheet.pdf. Additional information related to the BLE component 306 may be found at https://cdn-shop.adafruit.com/product-files/2267/MDBT4O-P256R.pdf. Although the example embodiments describe the use of a BLE wireless interface, it should be understood that other wireless interfaces, such as a WiFi interface (e.g., based on the IEEE 802.11 family of protocol standards), or other wireless transceivers based on wireless communication protocols known in the art, may be used in other embodiments.

Microcontroller Module Technical Specifications:
Measures 2.0″×0.9″×0.28″ (51 mm×23 mm×8 mm) without headers soldered in
Weight—5.7 grams
ATSAMD21G18@ 48 MHz with 3.3V logic/power
3.3V regulator with 500mA peak current output
USB native support, comes with USB bootloader and serial port debugging
20 GPIO pins
Hardware Serial, hardware I2C, hardware SPI support
8×PWM pins
10×analog inputs
1×analog output
Built in 100mA lipoly charger with charging status indicator LED
Pin #13 red LED for general purpose blinking
Power/enable pin
4 mounting holes
Reset button

An Adafruit HUZZAH32-ESP32 Microcontroller may be used in addition to or instead of the Feather MO Microcontroller described above. The HUZZAH32-ESP32 Microcontroller specifications are as follows:
240 MHz dual core Tensilica LX6 microcontroller with 600 DMIPS
Integrated 520 KB SRAM
Integrated 802.11b/g/n HT40 Wi-Fi transceiver, baseband, stack and LWIP
Integrated dual mode Bluetooth (classic and BLE)
4 MByte flash
On-board PCB antenna
Ultra-low noise analog amplifier
Hall sensor
10×capacitive touch interface
32 kHz crystal oscillator
3×UARTs (only two are configured by default in the Feather Arduino IDE support, one UART is used for bootloading/debug)
3×SPI (only one is configured by default in the Feather Arduino IDE support)
2×I2C (only one is configured by default in the Feather Arduino IDE support)
12×ADC input channels
2×I2S Audio
2×DAC
PWM/timer input/output available on every GPIO pin
OpenOCD debug interface with 32 kB TRAX buffer
SDIO master/slave 50 MHz
SD-card interface support

Also disposed within the device body 106 of the example embodiment is an Adafruit FeatherWing SD Card and Real-Time Clock Module (referred to herein as the “clock module 310”), as shown in FIG. 3B. The function of the clock module 310 is to (i) implement a real-time chronometer, (ii) generate a time-of-day code based on the chronometer, (iii) time-stamp each collected data sample with the time-of-day code, and (iv) store the time-stamped data on an associated data storage device (e.g., an SD card). The clock module 310 is powered by a dedicated energy source (e.g., a 3V CR1220 coin cell battery) to facilitate self-contained maintenance of the real-time chronometer in the absence of other energy sources. More information about the example clock module may be found at https://www.nxp.com/docs/en/data-sheet/PCF8523.pdf.

Also disposed within the device body 106 of the example embodiment is an Adafruit MPR121 12-channel capacitive touch sensor breakout module (referred to herein as the “touch sensor module 312”), as shown in FIG. 3C. The touch sensor module 312 enables the PROMOTE-KMC device to detect skin-to-skin contact between the parent and the baby, by evaluating signals from an electrode in contact with the parent and an electrode in contact with the baby. The electrodes may be implemented by a woven conductive fabric. General features of the touch sensor module 312 may include:
1.71V to 3.6V operation
29 μA typical run current at 16 ms sampling interval
3 μA in scan stop mode current
12 electrodes/capacitance sensing inputs in which 8 are multifunctional for LED driving and GPIO
Integrated independent autocalibration for each electrode input
Autoconfiguration of charge current and charge time for each electrode input
Separate touch and release trip thresholds for each electrode, providing hysteresis and electrode independence
I2C interface, with IRQ Interrupt output to advise electrode status changes
3 mm×3 mm×0.65 mm 20 lead QFN package
−40° C. to +85° C. operating temperature range

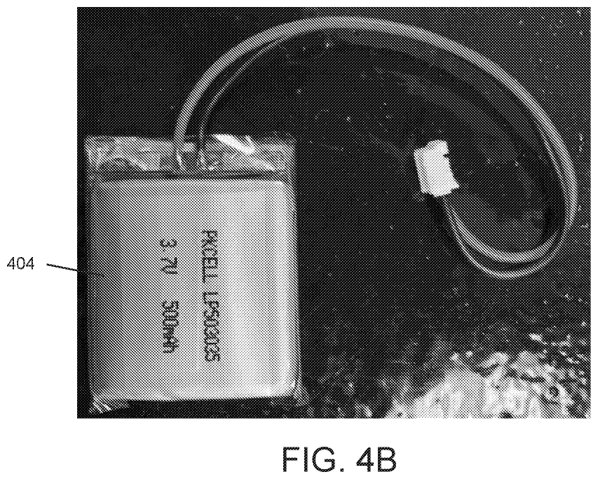

Also disposed within the device body 106 of the example embodiment is an Adafruit TMP007 Infrared Temperature Sensor (referred to herein as a “temperature module 402”), as shown in FIG. 4A. This temperature module 402 includes a temperature sensor component. The temperature sensor module measures the temperature of an object within a field of view of the temperature sensor. The temperature sensor is arranged to measure the skin temperature of the baby without requiring physical contact, and produces a digital temperature code. General features of the temperature module 402 may include:
Thermopile and Local Die Temperature Sensor
   Noise-equivalent temperature (NETD): 90 mK
   Responsivity: 9 V/W
   Sensor Noise: 300 nVrms.
Integrated Math Engine o 14-Bit (0.03125° C.) Resolution
   Alert Pin: Interrupt and Comparator Modes
   Nonvolatile Memory
   Programmable Conversion Rate
   Transient Correction
Low Quiescent Current: 270-μActive, 2-μA Shutdown
I2C and SMBus Compatible
8-Ball DSBGA, 1.9 mm×1.9 mm×0.625 mm Package

Also disposed within the device body 106 of the example embodiment is an LSM9DS1 9-degree of freedom (DOF) inertial measurement unit (IMU). This unit may be used for determining the position of the baby while the device is worn by that baby. The position of the baby may be determined with respect to the mother, and/or with respect to a predetermined reference frame. This sensor can measure acceleration, magnetometer and gyroscope values. The IMU provides a classic 3-axis accelerometer, which may determine which direction is down towards the Earth (i.e., by measuring gravity), or how fast the board is accelerating in three-dimensional (3D) space. The IMU also provides a 3-axis magnetometer that can determine a magnetic force gradient (e.g., to detect magnetic north). The IMU also provides a 3-axis gyroscope that may measure spin and twist.

Also disposed within the device body of the example embodiment is a heart rate sensor, which may be used to measure the heart rate of the baby during kangaroo-mother-care interaction with parent. Although a specific heart rate sensor is not described herein, such devices are well known in the art, and one skilled in the art would recognize that such a device would be readily available.

A device lid engages the device body 106, thereby enclosing the various electrical components within the device body 106, and isolating the electrical components from the external environment. The device lid of the example embodiment is printed with a 3D printer by using NinjaFlex Thermoplastic Urethane (TPU) filament, although other embodiments may utilize a lid fabricated by other techniques known in the art. Material properties and a safety document for the NinjaFlex TPU filament may be found at https://ninjatek.fppsites.com/wp-content/uploads/2018/10/NinjaFlex-TDS.pdf.

A flexible belt 104 attached to the device body 106 is configured to be wrapped around the baby, thereby maintaining physical contact between device body 106 and the baby. The belt 104 and the device body 106 is configured such that the temperature sensor 402 is directed toward the baby. The belt 104 also hosts the capacitive touch sensor 102, which facilitates detecting KMC interaction automatically. The belt 104 is printed with NinjaFlex TPU filament, the same material that is used to print the device lid.

The capacitive touch sensor 102 may be implemented with a woven conductive fabric, which is made of copper-nickel-plated nylon and it has a resistance of less than 1 ohm per foot in any direction across the textile. More information can be found at https://cdn-shop.adafruit.com/product-files/1168/Pn1168 Datasheet.pdf.

An energy source 404, for example a 500 mAh Lithium Polymer (li—po) battery, disposed within the device body, may provide power to the components of the PROMOTE-KMC device. The example energy source 404 (li—po battery), shown in FIG. 4B, may include a protection circuit to mitigate unexpected and potentially harmful issues (e.g., overcurrent events). More information about this specific li—po battery may be found at https://cdn-shop.adafruit.com/product-files/1578/1578+msds.pdf.

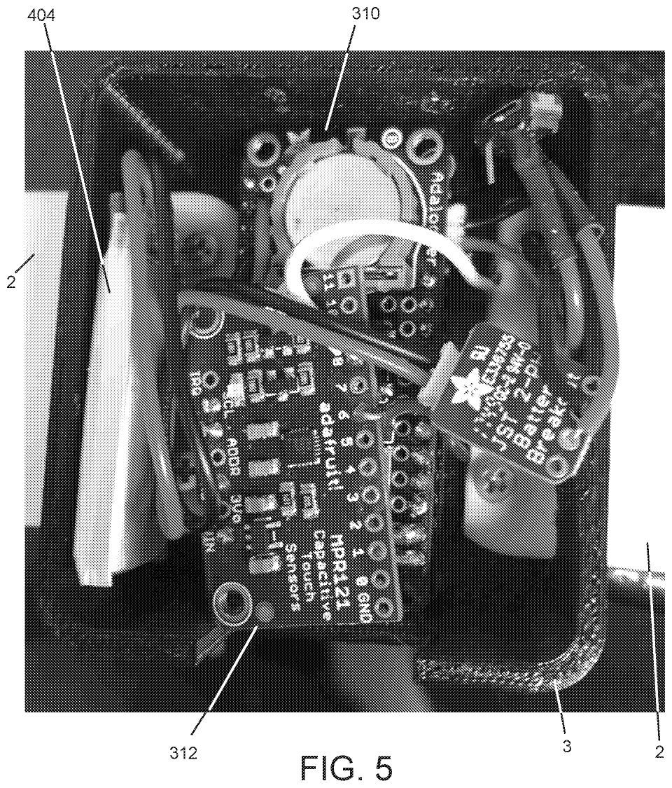

FIG. 5 shows a view of an example embodiment of the device body 106 with several of the components described herein situated in an example arrangement. Shown are the flexible belt 104, the device body 106, the clock module 310, the touch sensor module 312, and the energy source (battery) 404.

### Device Fabrication

Firstly, the electrical components described herein are electrically coupled to one another, as appropriate to implement the interconnections described, thereby forming an electrical unit. Secondly, the electrical unit is attached to device body. Thirdly, the conductive sensor fabrics 102 are mounted on the flexible belt 104, the device body 106 is attached to the flexible belt 104 and the conductive sensor fabrics 102 are electrically coupled to one or more components within the device body 106. Finally, device lid is attached to the top of the device body 106.

### Data Collection

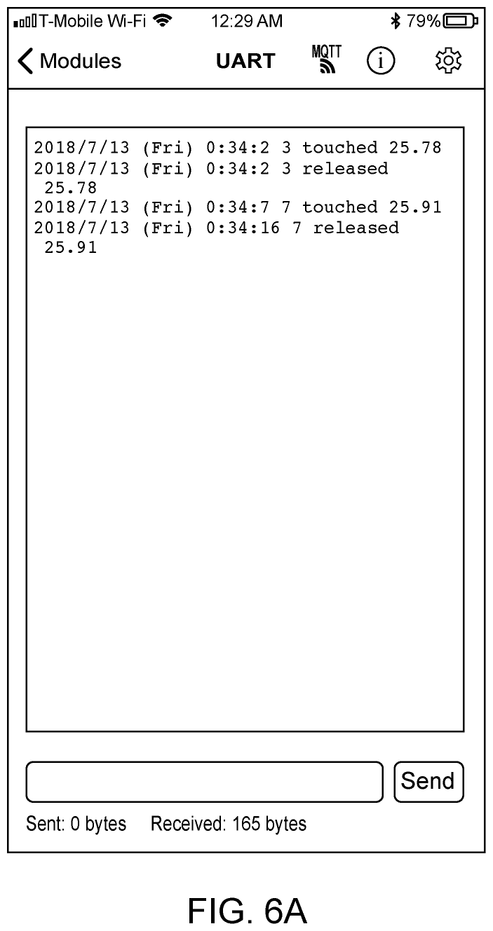

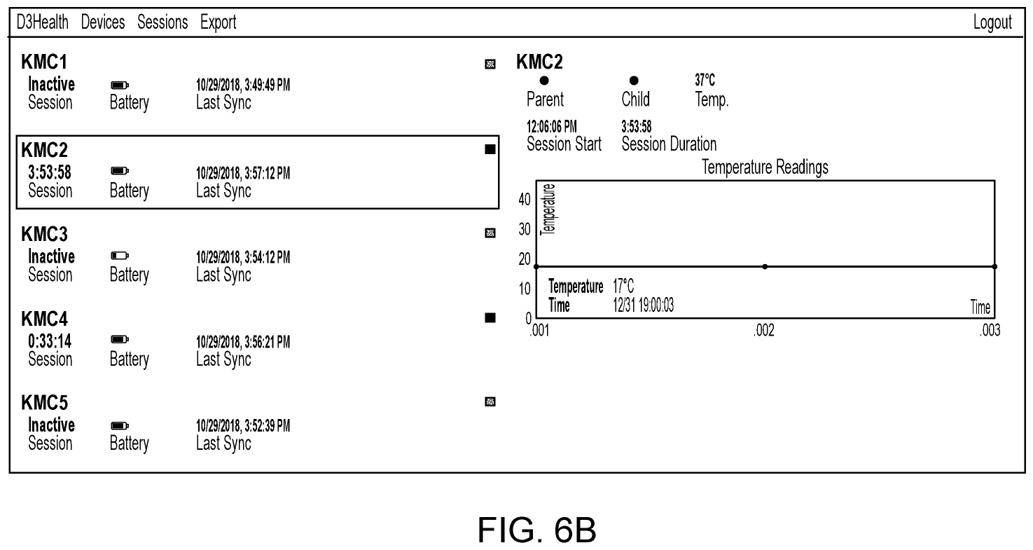

Data collected by the example PROMOTE-KMC device described herein may include, for example, frequency and duration of skin-to-skin contact between the neonate and the parents, and skin surface temperature from the neonate. Real-time information of data capture is also stamped to these collected data. The time-stamped data may be stored, for example, on an encrypted memory card. The stored data may be uploaded to an external storage facility, for example an HIPAA certified AWS Cloud infrastructure. Collected data also can be formatted to be viewed from a mobile device (e.g., smartphone or tablet) app and associated online cloud dashboard. FIG. 6A illustrates an example embodiment of a smartphone application displaying such collected data on a smartphone. FIG. 6B shows an example online dashboard, displayed in, for example, an Internet web browser, presenting such collected data. The dashboard of FIG. 6B shows five KMC devices (KMC1 through KMC5), two of which (KMC2 and KMC4) are showing active sessions. The dashboard shows KMC2 selected (by the box outlining that device entry), with the information specific to that device displayed on the right-most portion of the dashboard.

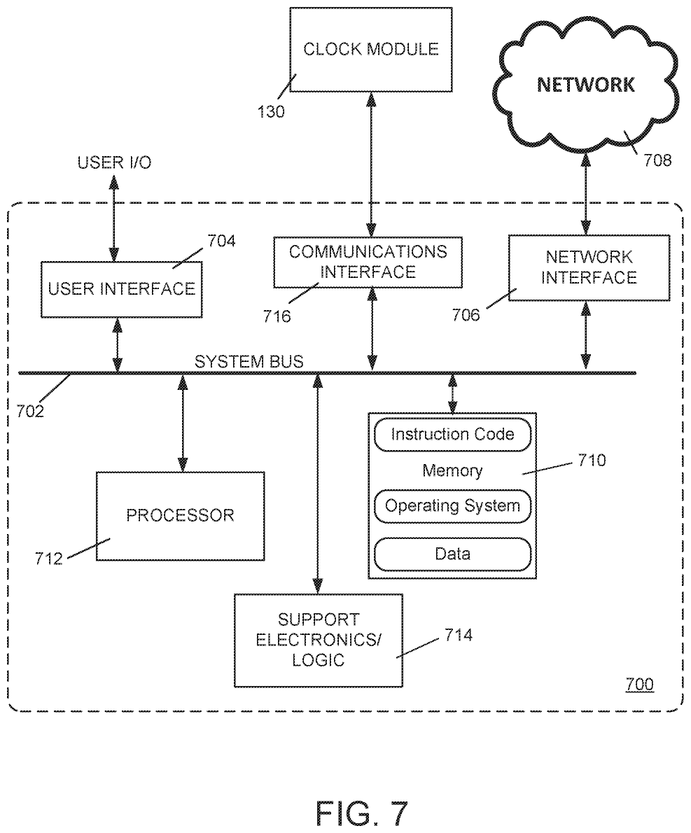

FIG. 7 is a diagram of an example internal structure of a processing system 700 that may be used to implement one or more of the embodiments herein. Each processing system 700 contains a system bus 702, where a bus is a set of hardware lines used for data transfer among the components of a computer or processing system. The system bus 702 is essentially a shared conduit that connects different components of a processing system (e.g., processor, disk storage, memory, input/output ports, network ports, etc.) that enables the transfer of information between the components.

Attached to the system bus 702 is a user I/O device interface 704 for connecting various input and output devices (e.g., keyboard, mouse, displays, printers, speakers, etc.) to the processing system 700. A network interface 706 allows the computer to connect to various other devices attached to a network 708. Memory 710 provides volatile and non-volatile storage for information such as computer software instructions used to implement one or more of the embodiments of the present invention described herein, for data generated internally and for data received from sources external to the processing system 700.

A central processor unit 712 is also attached to the system bus 702 and provides for the execution of computer instructions stored in memory 710. The system may also include support electronics/logic 714, and a communications interface 716. The communications interface may, for example, convey information to and/or from the clock module, described with reference to FIG. 4.

In one embodiment, the information stored in memory 710 may comprise a computer program product, such that the memory 710 may comprise a non-transitory computer-readable medium (e.g., a removable storage medium such as one or more DVD-ROM's, CD-ROM's, diskettes, tapes, etc.) that provides at least a portion of the software instructions for the invention system. The computer program product can be installed by any suitable software installation procedure, as is well known in the art. In another embodiment, at least a portion of the software instructions may also be downloaded over a cable communication and/or wireless connection as described herein.

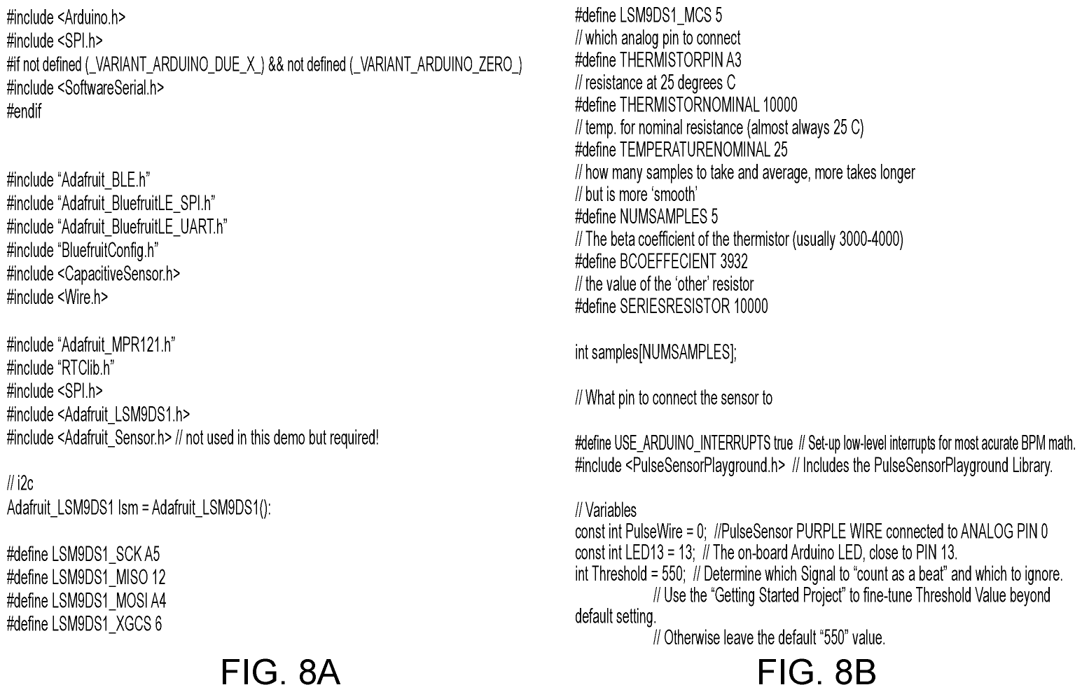

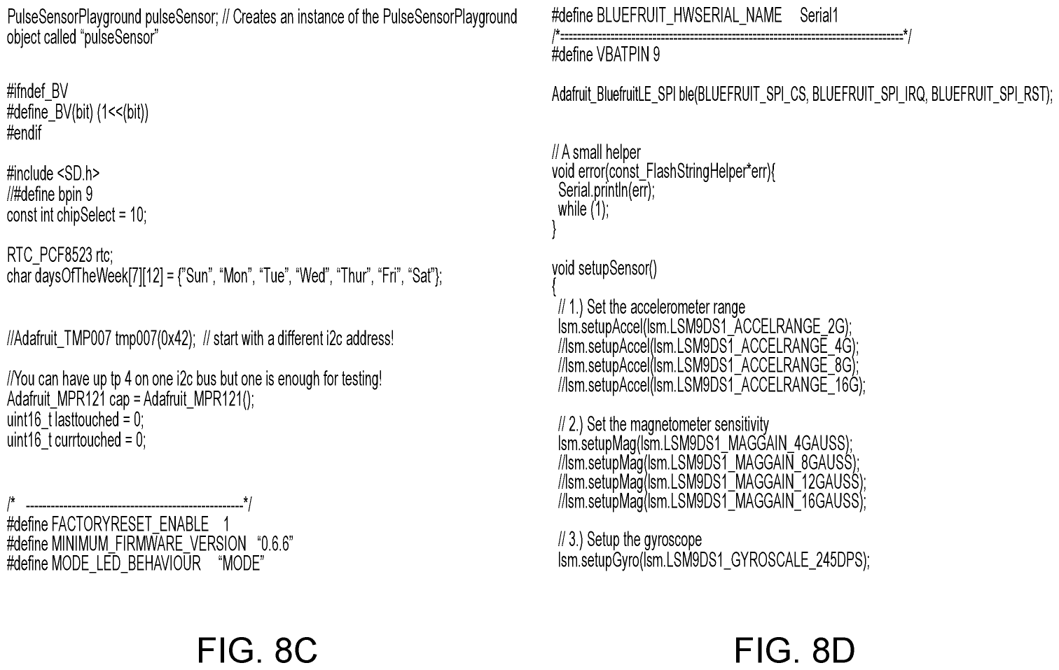

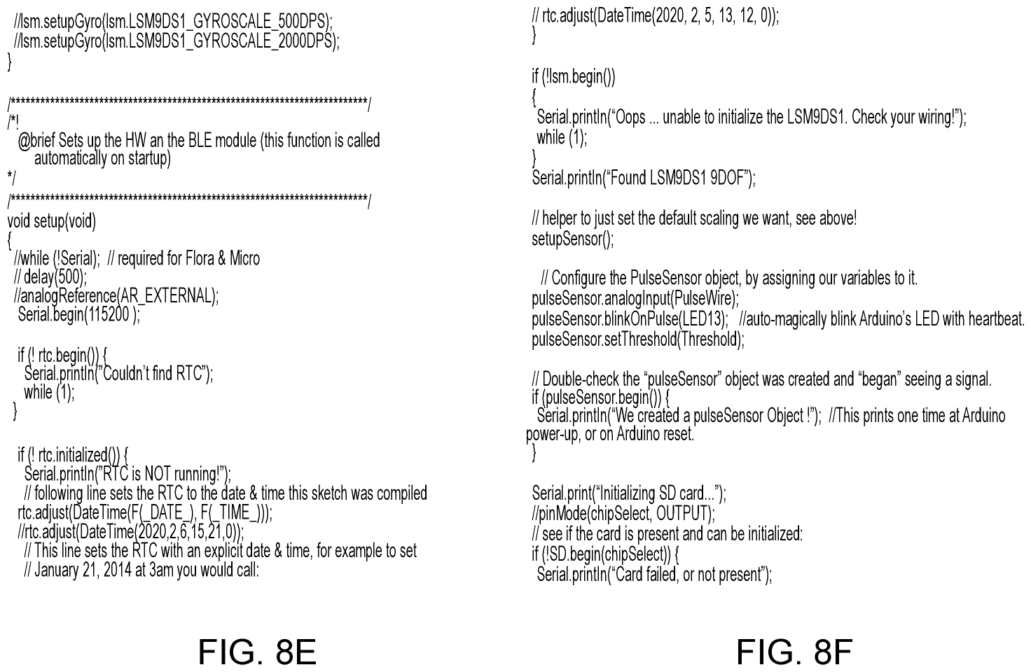

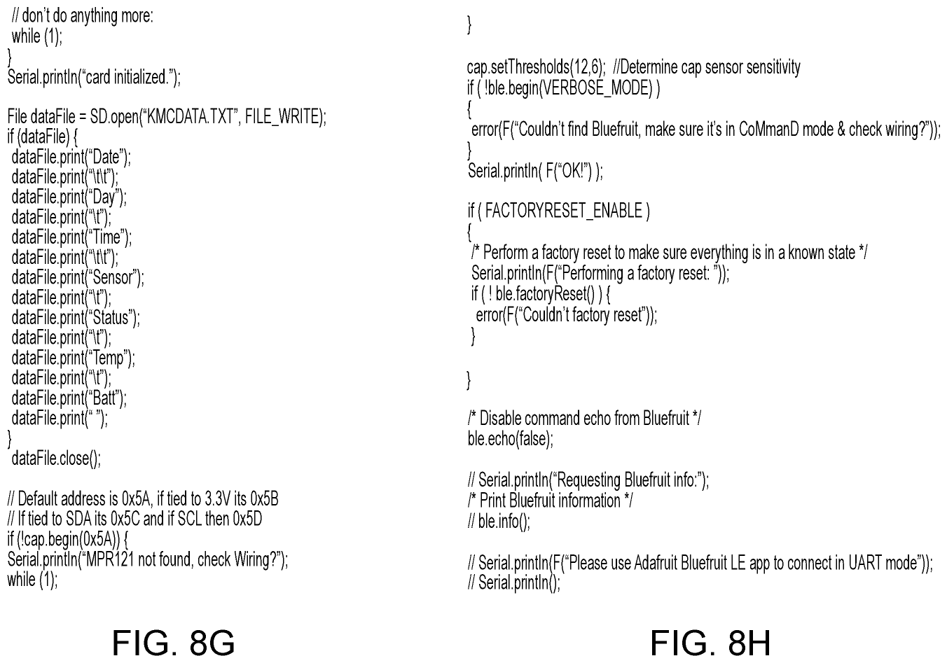

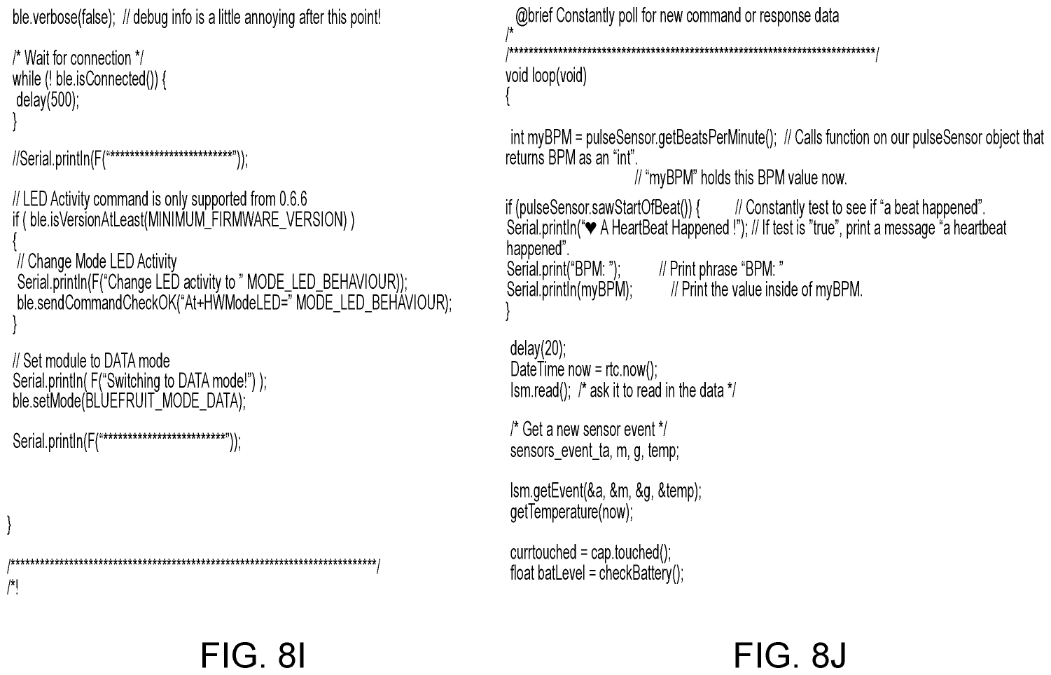

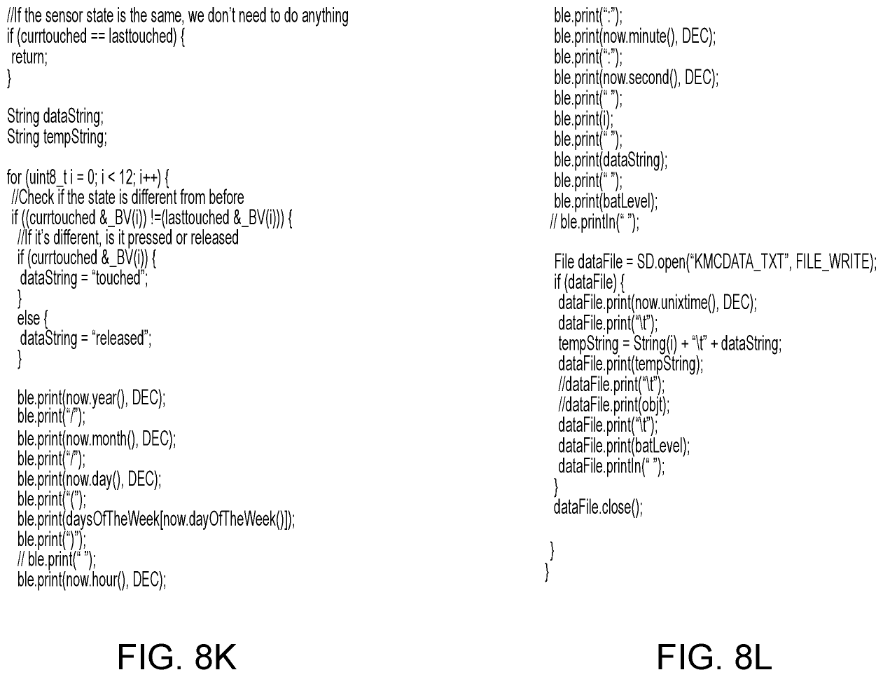

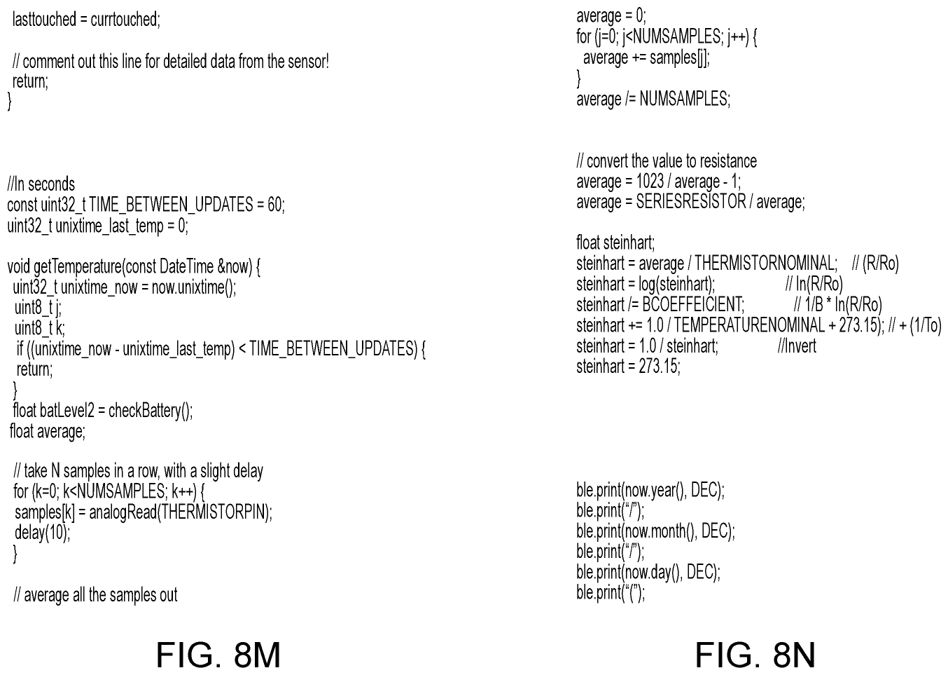

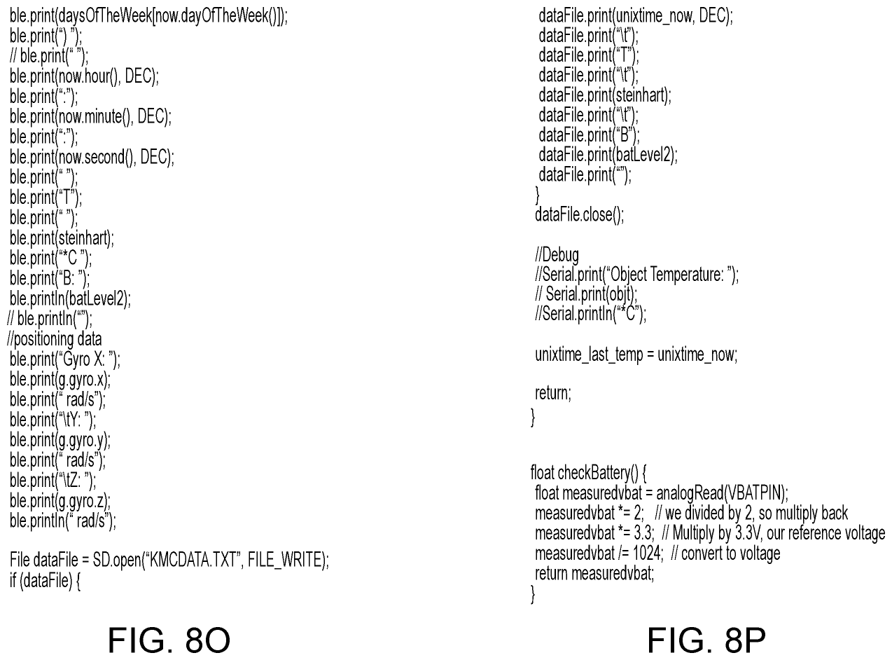

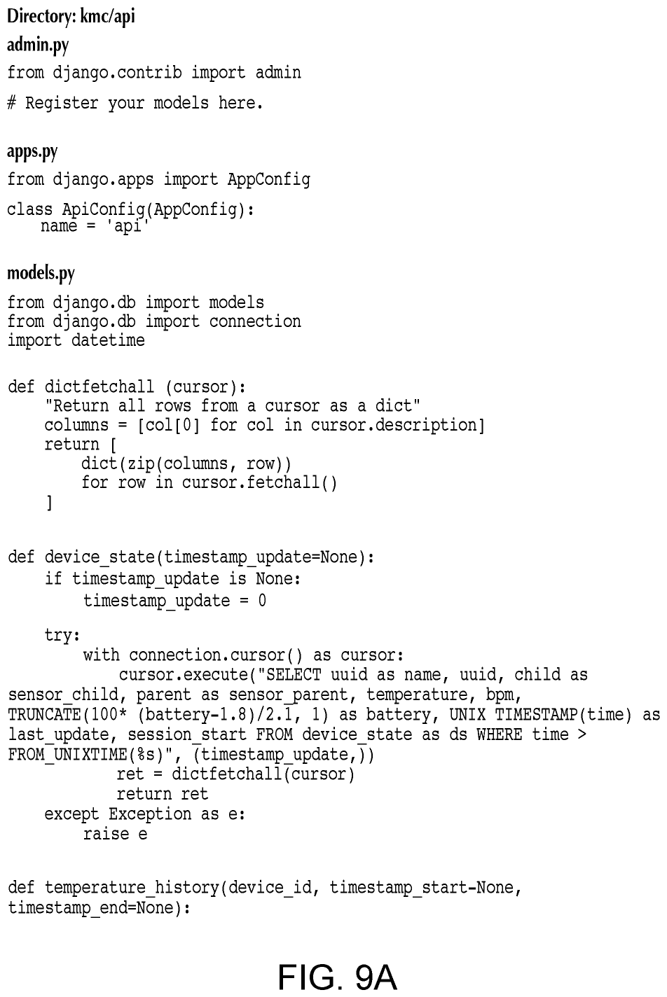

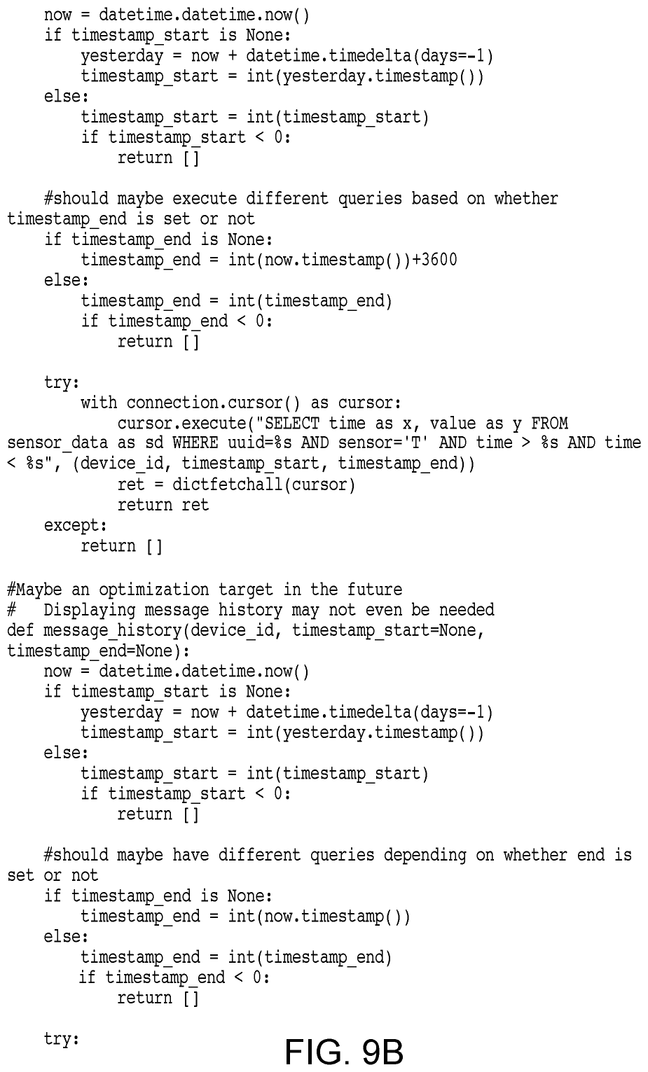

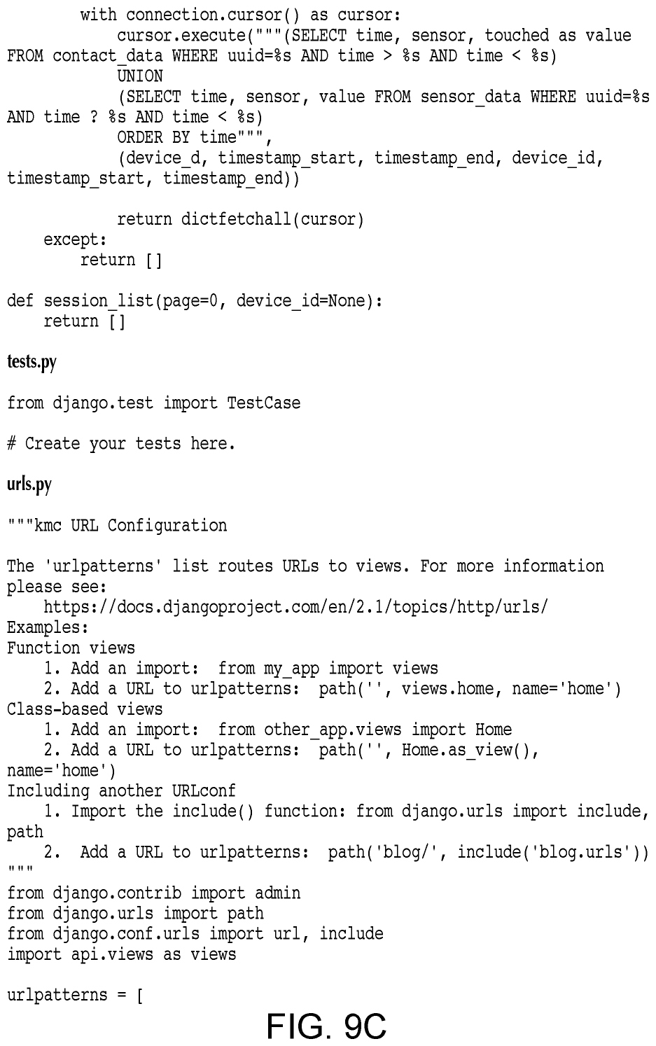

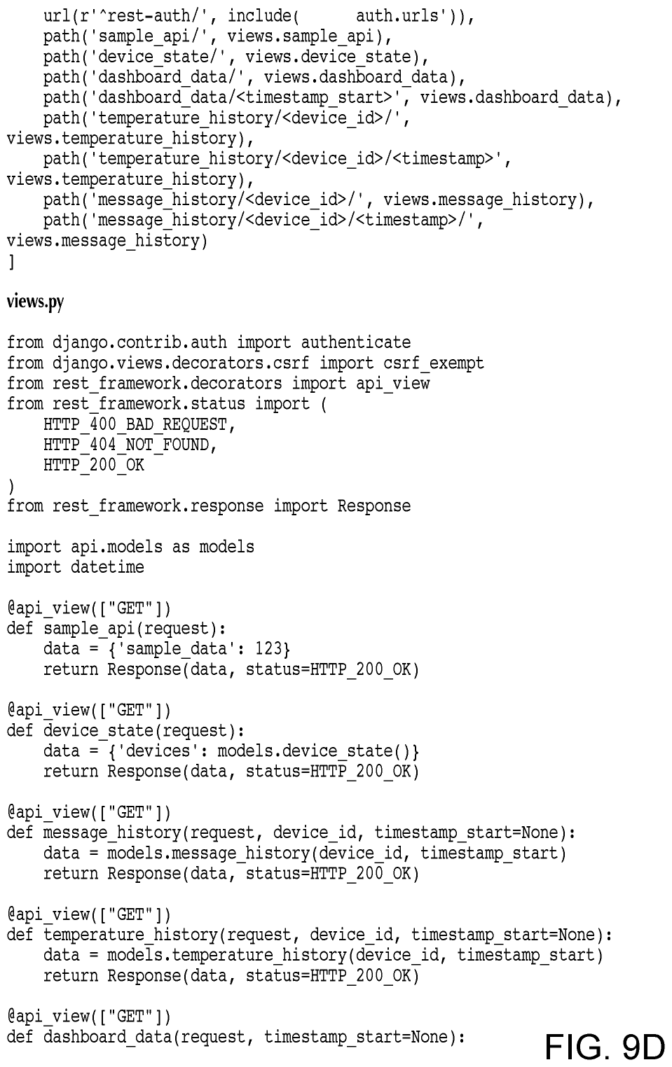

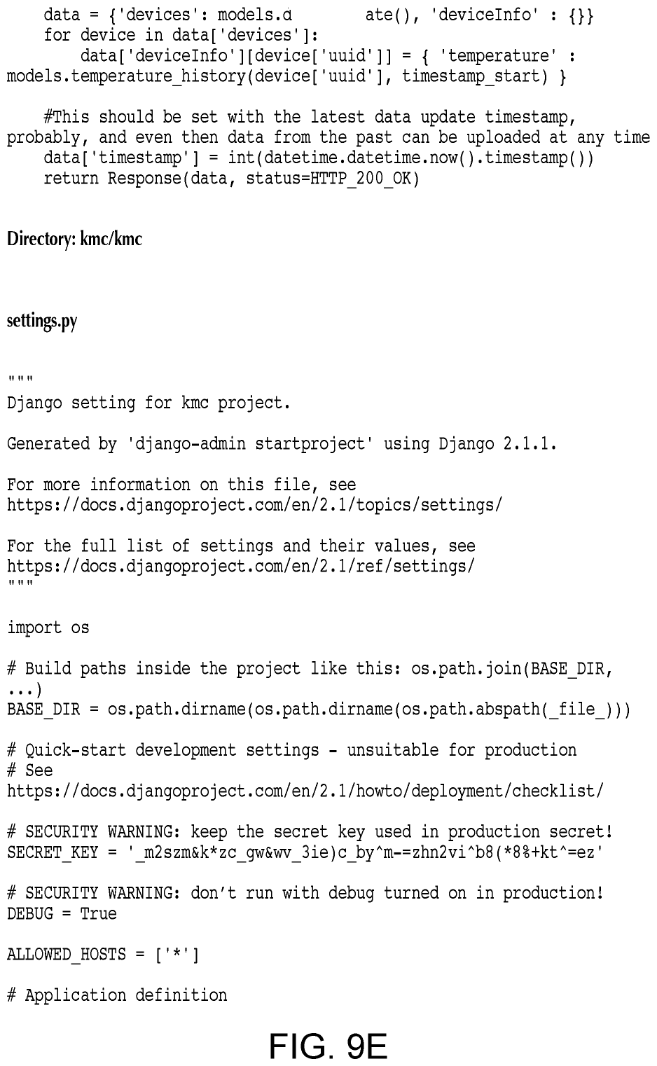

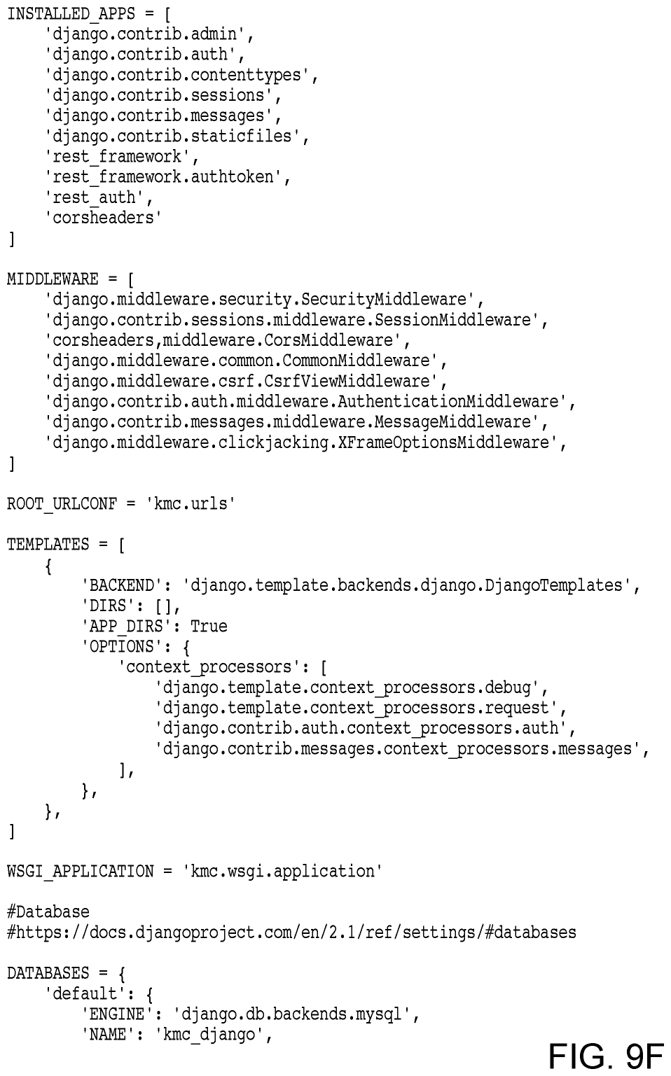

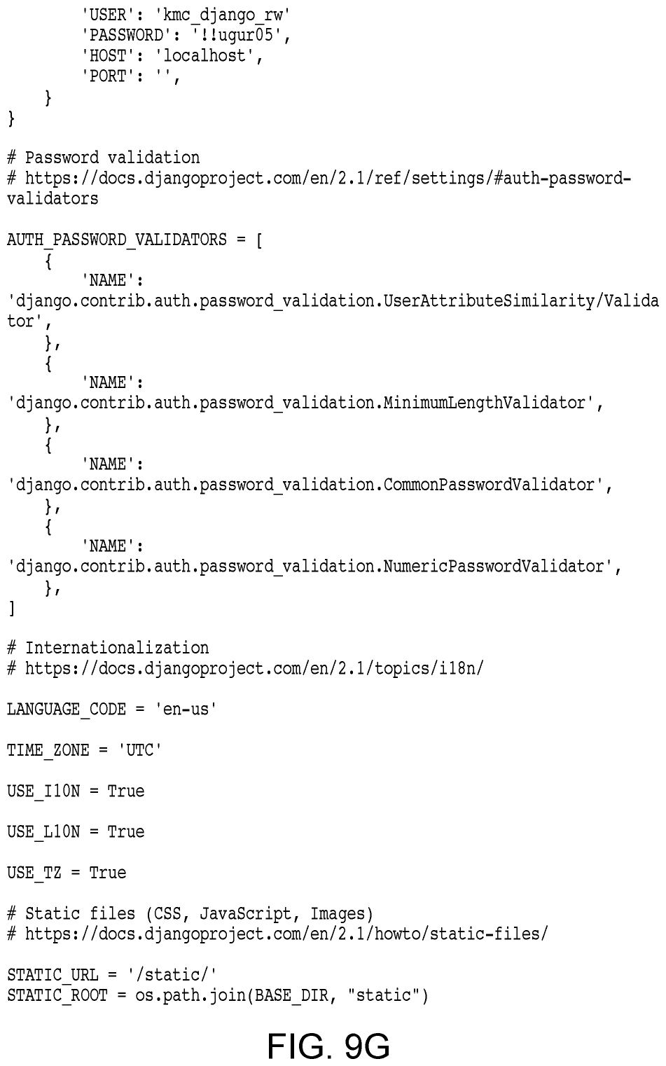

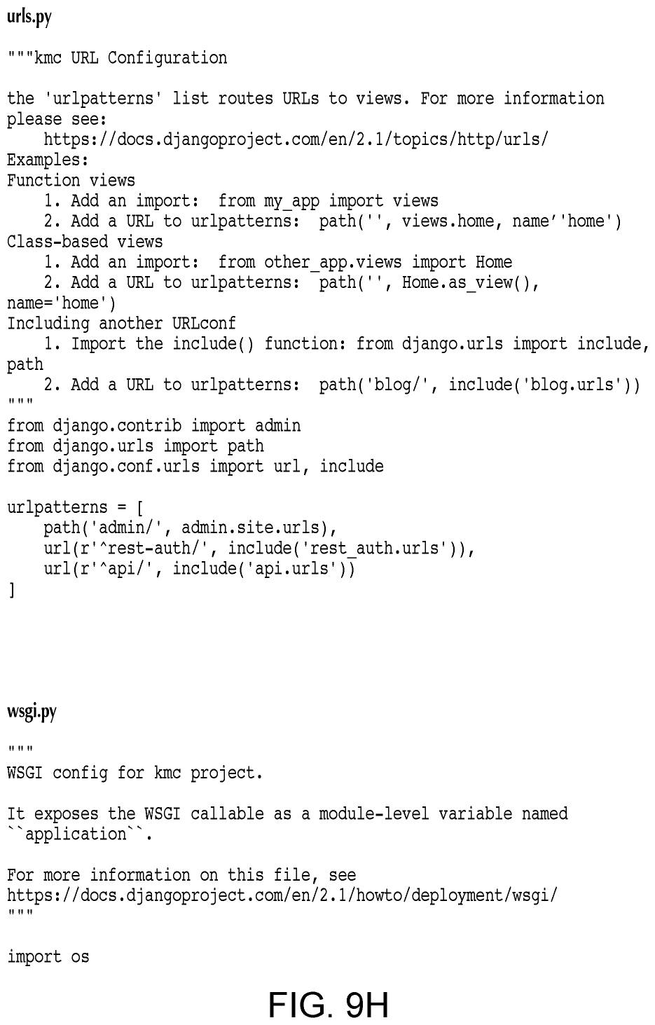

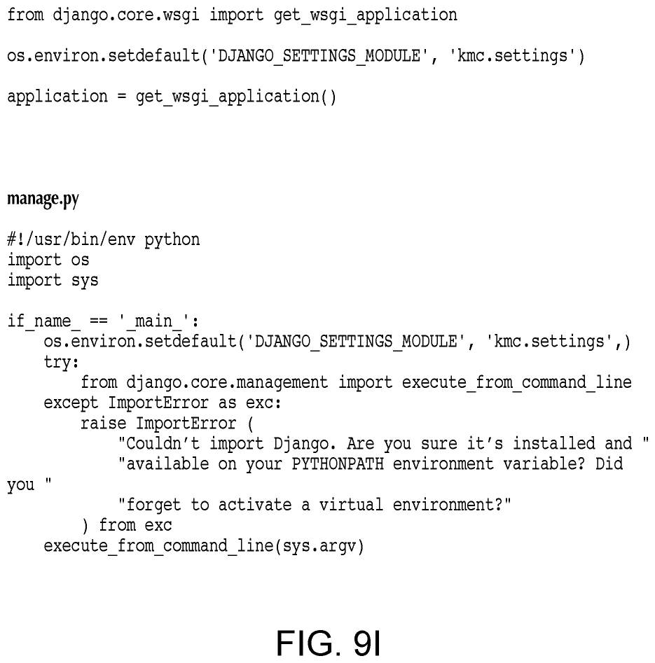

Example embodiments of software instructions, suitable for use in and with embodiments of the a PROMOTE-KMC device described herein, are presented in FIGS. 8A through 8P and FIGS. 9A through 9I. FIGS. 8A through 8P depict the example instruction code stored within the PROMOTE-KMC device and executed by the processor within the PROMOTE-KMC device to implement the functions and operations described herein. FIGS. 9A through 9I depict the example instruction code stored within the PROMOTE-KMC device and executed by the processor within the PROMOTE-KMC device to establish a communications link between the PROMOTE-KMC device and a cloud-based reporting application associated with the dashboard described herein, for example with respect to FIG. 6B.

For certain embodiments, the belt and device body may be fabricated from materials that meet certain biocompatibility standards (e.g., ISO 10993) for direct contact with intact skin. Factors taken into account may include, for example, cytotoxicity, sensitivity, and irritation. Embodiments may be fabricated in a clean room, and fabricated may follow suitable sanitization protocols. Some embodiments may include a belt size that is narrower and thinner than the example embodiments described herein. Further, the belt connection with device body may be arranged to produce a flush fit. For other embodiments, the belt closing mechanism may comprise a loop to adjust fit pursuant to the neonate's size. The device body may include one or more of an accessible on/off switch, an accessible charging port for the energy source (battery), grooves for the various constituent components to be anchored to limit mobility, and one or more visible LED or other suitable light sources. For example, a first light source may be provided to indicate that device is on and measuring data and a second light source may be provided to indicate skin-to-skin contact.

It will be apparent that one or more embodiments described herein may be implemented in many different forms of software and hardware. Software code and/or specialized hardware used to implement embodiments described herein is not limiting of the embodiments of the invention described herein. Thus, the operation and behavior of embodiments are described without reference to specific software code and/or specialized hardware—it being understood that one would be able to design software and/or hardware to implement the embodiments based on the description herein.

Further, certain embodiments of the example embodiments described herein may be implemented as logic that performs one or more functions. This logic may be hardware-based, software-based, or a combination of hardware-based and software-based. Some or all of the logic may be stored on one or more tangible, non-transitory, computer-readable storage media and may include computer-executable instructions that may be executed by a controller or processor. The computer-executable instructions may include instructions that implement one or more embodiments of the invention. The tangible, non-transitory, computer-readable storage media may be volatile or non-volatile and may include, for example, flash memories, dynamic memories, removable disks, and non-removable disks.

While example embodiments have been particularly shown and described, it will be understood by those skilled in the art that various changes in form and details may be made therein without departing from the scope of the embodiments encompassed by the appended claims.

What is claimed is:

1. An apparatus for measurement of skin-to-skin contact between a neonate and a parent of the neonate, comprising:
a capacitive touch sensor module configured to receive signals from a first electrode and a second electrode, and produce detected contact information associated with at least one of the first electrode and the second electrode;
a temperature module having a temperature sensor, the temperature module configured to measure a temperature of an object within a field of view of the temperature sensor, and to generate a corresponding temperature code;
a clock module configured to (i) implement a real-time chronometer, (ii) generate a time-of-day code based on the chronometer, (iii) time-stamp each collected data sample with the time-of-day code, each collected data sample comprising the detected contact information and the temperature code, and (iv) store the time-stamped data sample on an associated data storage device;
a processor; and
a memory with computer code instructions stored thereon, the memory operatively coupled to the processor such that, when executed by the processor, the computer code instructions cause the apparatus to coordinate operation of the capacitive touch sensor module, the temperature module, and the clock module.

2. The apparatus of claim 1, wherein the touch sensor module, the temperature module, the clock module, the processor and the memory are disposed within a housing that comprises a device body and device lid, and wherein the device body and the device lid are configured to engage one another to isolate the touch sensor module, the temperature module, the clock module, the processor and the memory from an external environment.

3. The apparatus of claim 2, wherein the housing is attached to a flexible belt, the first electrode is disposed on a first side of the flexible belt, and the second electrode is disposed on a second side of the belt.

4. The apparatus of claim 1, wherein the first electrode is configured to be in physical contact with skin of the neonate, the second electrode is configured to be in physical contact with skin of the parent of the neonate, and the apparatus is operative to characterize aspects of skin-to-skin contact between the neonate and the parent of the neonate.

5. The apparatus of claim 1, further comprising a wireless transceiver operatively coupled to the processor, the wireless transceiver configured to wirelessly communicate information from the apparatus to an external peripheral component.

6. The apparatus of claim 5, wherein the wireless transceiver is one of a Bluetooth Low Energy (BLE) transceiver or a WiFi transceiver. The apparatus of claim 1, further comprising an energy source configured to provide electrical energy to the touch sensor module, the temperature module, the clock module, the processor and the memory.

8. The apparatus of claim 1, further comprising an inertial measurement unit configured to determine a position of the neonate with respect to one or both of (i) the parent and (ii) a predetermined reference frame.

9. A method of measuring skin-to-skin contact between a neonate and a parent of the neonate, comprising:
providing a flexible belt for disposing around the neonate, such that a first electrode attached to a first side of the flexible belt is arranged to be in contact with the neonate, and a second electrode attached to a second side of the flexible belt is arranged to be in contact with the parent of the neonate;
receiving, by a capacitive touch sensor module disposed within a housing attached to the flexible belt, information associated with at least one of the first electrode and the second electrode;
measuring, by a temperature module, a temperature of the neonate and generating a corresponding temperature code;
time-stamping, by a clock module, one or both of the information associated with at least one of the first electrode and the second electrode and the temperature code to produce time stamped information, and storing the time stamped information on an associated data storage device.

10. The method of claim 9, further comprising:
a) measuring physiological parameters, consisting of one or more of
i) heart rate of the neonate,
ii) respiratory rate of the neonate,
iii) sympathetic activity of the neonate, and
iv) positioning of the neonate with respect to the parent of the neonate;
b) time-stamping the physiological parameters; and
c) storing the time stamped physiological parameters on an associated data storage device.

11. The method of claim 9, further comprising wirelessly transmitting the time stamped information to a destination that is external to the housing.

12. The method of claim 11, further comprising displaying, at the destination, a dashboard that presents the time stamped information to a user.

13. The method of claim 9, further comprising measuring one or both of a temperature and a heart rate of the neonate before an indication of neonate-to-parent skin-to-skin contact, and measuring one or both of the temperature and the heart rate of the neonate after the indication of neonate-to-parent skin-to-skin contact.

14. An apparatus for measurement of skin-to-skin contact between a neonate and a parent of the neonate, comprising:
a touch sensor that receives signals from a first electrode and a second electrode, and produces detected contact information associated with at least one of the first electrode and the second electrode;
a temperature sensor that measures a temperature of an object within a field of view of the temperature sensor, and generates a corresponding temperature code;
a data accumulator that (i) applies a time-of-day code time stamp to each collected data sample, where each collected data sample comprises the detected contact information and the temperature code, and (ii) stores each time-stamped data sample on an associated data storage device.

15. The apparatus of claim 14, further comprising an inertial measurement unit configured to determine a position of the neonate with respect to one or both of (i) the parent and (ii) a predetermined reference frame.

16. The apparatus of claim 14, wherein the touch sensor, the temperature sensor, and the data accumulator are disposed within a housing that is configured to isolate the touch sensor module, the temperature module, and the data accumulator from an external environment.

17. The apparatus of claim 16, wherein the housing is attached to a flexible belt, the first electrode is disposed on a first side of the flexible belt, and the second electrode is disposed on a second side of the belt.

18. The apparatus of claim 14, further comprising a wireless transceiver configured to wirelessly communicate information from the apparatus to an external peripheral component.

19. The apparatus of claim 18, wherein the wireless transceiver is a Bluetooth Low Energy (BLE) transceiver.

20. The apparatus of claim 14, further comprising an energy source configured to provide electrical energy to the touch sensor module, the temperature module, and the data accumulator.
